SEQUENCE LISTING (12 sequences)

SEQ ID NO: 1 - DNA, 21 bp, synthetic construct
  source 1..21: PCR_primers = fwd_name:F,fwd_seq: cggcccgctttataggcgttg,re v_name:R,rev_seq:cccactcgtcccgtccatcg; mol_type = other DNA; note = PCR primer F
cggcccgctttataggcgttg

SEQ ID NO: 2 - DNA, 20 bp, synthetic construct
  source 1..20: PCR_primers = fwd_name:F,fwd_seq: cggcccgctttataggcgttg,re v_name:R,rev_seq:cccactcgtcccgtccatcg; mol_type = other DNA; note = PCR primer R
cccactcgtcccgtccatcg

SEQ ID NO: 3 - DNA, 20 bp, synthetic construct
  source 1..20: PCR_primers = fwd_name:env_F,fwd_seq: ggatgaggtgactaagaaag ,rev_name:env_R,rev_seq:agagaaagaggggtgtctaaggaga; mol_type = other DNA; note = primer env-F for RT-PCR amplification of ALV-A-env
ggatgaggtgactaagaaag

SEQ ID NO: 4 - DNA, 25 bp, synthetic construct
  source 1..25: PCR_primers = fwd_name:env_F,fwd_seq: ggatgaggtgactaagaaag ,rev_name:env_R,rev_seq:agagaaagaggggtgtctaaggaga; mol_type = other DNA; note = primer env-R for RT-PCR amplification of ALV-A-env
agagaaagaggggtgtctaaggaga

SEQ ID NO: 5 - DNA, 21 bp, synthetic construct
  source 1..21: PCR_primers = fwd_name:env_F,fwd_seq: gcaccaccttgggaactgac c,rev_name:env_R,rev_seq:ggcgtggatcgacagcacac; mol_type = other DNA; note = primer env-F for RT-PCR amplification of ALV-K-env
gcaccaccttgggaactgacc

SEQ ID NO: 6 - DNA, 20 bp, synthetic construct
  source 1..20: PCR_primers = fwd_name:env_F,fwd_seq: gcaccaccttgggaactgac c,rev_name:env_R,rev_seq:ggcgtggatcgacagcacac; mol_type = other DNA; note = primer env-R for RT-PCR amplification of ALV-K-env
ggcgtggatcgacagcacac

SEQ ID NO: 7 - DNA, 23 bp, synthetic construct
  source 1..23: PCR_primers = fwd_name:P1_F,fwd_seq: gttcagcagatcctcatctcc cg,rev_name:P1_R,rev_seq:ggccattgtgcgatctaagaggg; mol_type = other DNA; note = primer P1-F for PCR amplification of Fragment 1
gttcagcagatcctcatctcccg

SEQ ID NO: 8 - DNA, 23 bp, synthetic construct
  source 1..23: PCR_primers = fwd_name:P1_F,fwd_seq: gttcagcagatcctcatctcc cg,rev_name:P1_R,rev_seq:ggccattgtgcgatctaagaggg; mol_type = other DNA; note = primer P1-R for PCR amplification of Fragment 1
ggccattgtgcgatctaagaggg

SEQ ID NO: 9 - DNA, 20 bp, synthetic construct
  source 1..20: PCR_primers = fwd_name:P2_F,fwd_seq: agccctcttagatcgcacaa, rev_name:P2_R,rev_seq:gtgacaccgagcacaaaatg; mol_type = other DNA; note = primer P2-F for PCR amplification of Fragment 2
agccctcttagatcgcacaa

SEQ ID NO: 10 - DNA, 20 bp, synthetic construct
  source 1..20: PCR_primers = fwd_name:P2_F,fwd_seq: agccctcttagatcgcacaa, rev_name:P2_R,rev_seq:gtgacaccgagcacaaaatg; mol_type = other DNA; note = primer P2-R for PCR amplification of Fragment 2
gtgacaccgagcacaaaatg

SEQ ID NO: 11 - DNA, 20 bp, synthetic construct
  source 1..20: PCR_primers = fwd_name:P3_F,fwd_seq: gttggagctggatgagcact, rev_name:P3_R,rev_seq:tgagggaattcctgtcacct; mol_type = other DNA; note = primer P3-F for PCR amplification of Fragment 3
gttggagctggatgagcact

SEQ ID NO: 12 - DNA, 20 bp, synthetic construct
  source 1..20: PCR_primers = fwd_name:P3_F,fwd_seq: gttggagctggatgagcact, rev_name:P3_R,rev_seq:tgagggaattcctgtcacct; mol_type = other DNA; note = primer P3-R for PCR amplification of Fragment 3
tgagggaattcctgtcacct

(19) United States
(12) Patent Application Publication
(10) Pub. No.: US 2023/0313319 A1
(43) Pub. Date: Oct. 5, 2023

(54) MOLECULAR MARKER FOR GENETIC RESISTANCE OF CHICKEN TO INFECTION BY SUBGROUPS A AND K AVIAN LEUKOSIS VIRUS AND USE THEREOF

(71) Applicant: South China Agricultural University, Guangzhou City (CN)

(72) Inventors: Qingmei XIE, Guangzhou City (CN); Weiguo CHEN, Guangzhou City (CN); Wenxue LI, Guangzhou City (CN); Yu NIE, Guangzhou City (CN); Liqin LIAO, Guangzhou City (CN); Xinheng ZHANG, Guangzhou City (CN)

(73) Assignee: South China Agricultural University, Guangzhou City (CN)

(21) Appl. No.: 18/192,107

(22) Filed: Mar. 29, 2023

(30) Foreign Application Priority Data
Mar. 30, 2022 (CN) 202210321583.2

(51) Int. Cl. C12Q 1/6888

(52) U.S. Cl. CPC C12Q 1/6888; C12Q 2600/124; C12Q 2600/156

(57) ABSTRACT

Disclosed is a molecular marker for genetic resistance of chicken to infection by subgroups A and K avian leukosis virus (ALV-A and ALV-K) and use thereof the molecular marker is tva gene with base deletion between 318-323 and/or between 602-607; specifically, bases ACCTCC at positions 318-323 and bases CCGCTG at positions 602-607 are deleted. In the present disclosure, genetic variation of tva receptor gene in Chinese chicken breeds is analyzed, and it is found that the DNA sequence of tva receptor gene in the Chinese chicken breeds has base deletion at positions 318-323 or at positions 602-607. Moreover, a method for breeding of the chicken breeds resistance to ALV-A and ALV-K has been established.

### CROSS REFERENCE TO RELATED APPLICATION

This patent application claims the benefit and priority of Chinese Patent Application No. 202210321583.2 filed with the China National Intellectual Property Administration on Mar. 30, 2022, the disclosure of which is incorporated by reference herein in its entirety as part of the present application.

### REFERENCE TO SEQUENCE LISTING

A computer readable XML file entitled “AttachB_SequenceListing-0436A”, that was created on Jul. 24, 2023, with a file size of about 22 kb, contains the sequence listing for this application, has been filed with this application, and is hereby incorporated by reference in its entirety.

### TECHNICAL FIELD

The present disclosure belongs to the technical field of selective breeding of disease-resistant poultry breeds, and in particular to molecular markers for genetic resistance of chicken to infection by subgroups A and K avian leukosis virus (ALV-A and ALV-K) and use thereof.

### BACKGROUND

Avian leukosis (AL) is an avian immunosuppressive neoplastic infectious disease caused by avian leukosis virus (ALV). Currently, AL has become the most serious disease that endangers the safety of modern poultry breeding industry. ALV that can naturally infect chickens includes seven subgroups, namely, ALV-A to ALV-E, ALV-J, and ALV-K. Among them, ALV-A and ALV-K are main pathogens causing the AL of chickens in China. ALV-A and ALV-K mainly infect offspring through vertical transmission, with infection capable of being gradually amplified in a linkage of “great-grandparent, grandparent, parent, and commercial generation”, at an infection rate expanding by about 5% to 20% in each generation. Infection with ALV-A or ALV-K in one great-grandparent breeding hen may lead to infection in 240,000 commercial broilers. ALV-A and ALV-K infection can lead to the death of chickens with characteristic tumors, cause a decreased production performance, and result in severe immunosuppression in infected chickens. As a result, other viral and bacterial diseases such as avian influenza and newcastle disease are easily concomitant or secondary infection, causing huge economic losses to the poultry industry.

Currently, there is no commercial vaccine or effective treatment for AL caused by ALV-A and ALV-K. The traditional method for the control of AL is mainly by eliminating positive chickens and purifying breeder flocks, but this method has the following disadvantages: (1) long purification time: it requires 3 to 5 generations in 5 to 8 years to purify a breeder flock; (2) high labor intensity: each breeder should be tested 4 to 6 times by enzyme-linked immunosorbent assay (ELISA) and virus isolation in each generation; (3) high cost: it costs about 500 yuan in ALV testing and purification for each chicken, and an annual cost of AL purification in China exceeds 1 billion yuan; and (4) negative chickens after purification still have a risk of re-infection with ALV, and the purification effect is easy to relapse, resulting in a continuous struggle between pathogens (ALV-A and ALV-K), animals, and quarantine officers. In recent years, epidemiological investigations of ALV have found that ALV-A and ALV-K are prevalent in local chicken breeds, commercial broilers, laying hens, and wild birds in China.

It can be seen that the method of population purification cannot completely control the occurrence and prevalence of ALV-A and ALV-K-caused AL in Chinese chickens. Therefore, it is extremely urgent to explore and identify molecular markers for genetic resistance to ALV-A and ALV-K, improve the genetic resistance of host to infection by ALV-A and ALV-K, and develop the new strategy and method that is more suitable for the control of AL in China.

### SUMMARY

In view of the above deficiencies, molecular markers for genetic resistance of chicken to ALV-A and ALV-K and use thereof are provided in the present disclosure. The technical scheme of the present disclosure overcomes the prior-art deficiencies that the occurrence and prevalence of ALV-A and ALV-K cannot be completely controlled in Chinese chickens.

To achieve the above objective, the present disclosure adopts the following technical schemes:

Molecular markers for genetic resistance of chicken to infection by ALV-A and ALV-K, wherein the molecular marker is tva gene with base deletion between 318-323 and/or between 602-607; specifically.

Bases ACCTCC at positions 318-323 and bases CCGCTG at positions 602-607 are deleted.

In the present disclosure, genetic variation of tva receptor gene of Chinese chicken breeds (including a total of 6,570 blood samples from 28 local chicken breeds and 57 yellow feathered broiler lines) is analyzed. It is found that in a DNA sequence of tva receptor gene (with a GenBank accession number of AY531262.1) in Chinese chicken breeds, a deletion mutation of ACCTCC at positions 318-323 may exist, and a deletion mutation of CCGCTG at positions 602-607 may exist. The two deletion mutation sites are abbreviated as tva^{318-323delACCTCC} and tva^{602-607delCCGCTG} mutation sites.

In addition, it is confirmed from in vitro and in vivo experiments that the natural mutation of tva gene causes the host to be resistant to ALV-A and ALV-K infection. The reason is that Tva is a low-density lipoprotein receptor (LDLR), and there is an LDL-A repeat motif including 40 amino acid residues between 11th to 50th amino acid residues in an extracellular region of the Tva protein. The LDL-A repeat motif is rich in cysteine, and 3 essential cysteine disulfide bonds are formed between 6 cysteine residues, which is a key region that mediates the infection of ALV-A and ALV-K to host cells.

The tva^{318-323delACCTCC} mutation is located in exon 1 of the tva receptor gene, which is located at positions 61-66 of tva gene coding sequence CDS (a tva gene mRNA reference sequence is NM_001044645.1), resulting in deletion of amino acids at positions 21-22 of the Tva receptor protein (a Tva receptor protein reference sequence is NP_001038110.1). It is speculated that the tva^{318-323delACCTCC} mutation causes tva receptor gene to express a functionally-defective Tva receptor protein with 2 key amino acids deleted in a signal peptide region, thereby causing the host to generate genetic resistance to infection by ALV-A and ALV-K.

In addition, the tva^{602-607delCCGCTG} mutation causes the deletion of CCGCTG at positions 151-156 of tva gene coding sequence CDS (the tva gene mRNA reference sequence is NM_001044645.1), resulting in deletion of amino acid at position 30 (proline, P) and deletion of amino acid at position 31 (leucine, L) in the extracellular region of the Tva receptor protein. It is speculated that the tva^{602-607delCCGCTG} mutation causes tva gene to express a defective Tva receptor protein, thereby causing the host to generate resistance to the ALV-A and ALV-K infection.

The present disclosure further provides primers for detecting the molecular marker, nucleotide sequences of the primers are set forth in SEQ ID NO: 1 and SEQ ID NO: 2.

The present disclosure further provides use of the molecular marker or the primers in screening/identifying chicken resistance to ALV-A and ALV-K.

Further, the use includes the following steps:
(1) extracting a genomic DNA of a sample to be tested, amplifying a tva gene fragment containing tva^{318-323delACCTCC} and/or tva^{602-607delCCGCTG} deletion sites with the primers set forth in SEQ ID NO: 1 and SEQ ID NO: 2, and sequencing to determine whether the sample to be tested is a resistant chicken; and
(2) if there are homozygous deletion mutations (tva^{delACCTCC/delACCTCC} and tva^{delCCGCTG/delCCGCTG}) at positions 318-323 and/or 602-607 in tva gene DNA sequence (with a GenBank accession number of AY531262.1), determining the sample to be tested has a phenotype of genetic resistance to the ALV-A and ALV-K infection; in other words, if a chicken to be tested has a genotype of tva^{delACCTCC/delACCTCC} or tva^{delCCGCTG/delCCGCTG}, or the base deletion exists simultaneously at positions 318-323 and 602-607, determining the chicken to be tested as chicken resistance to ALV-A and ALV-K;
if there is no deletion mutation at positions 318-323 and 602-607 of the DNA sequence of tva gene, determining the sample to be tested as a wild-type, which is susceptible to ALV-A and ALV-K infection (no resistance); that is, if a chicken to be tested has a genotype of wild-type tva^{s/s}, determining the chicken to be tested as chicken susceptible to the ALV-A and ALV-K; and
if there is a heterozygous deletion mutation (tva^{s/delACCTCC} and tva^{s/delCCGCTG}) at positions 318-323 and/or 602-607 of the DNA sequence of tva gene, determining the sample to be tested as chicken susceptible to the ALV-A and ALV-K.

However, individuals with the genotype of tva^{delACCTCC/delACCTCC} and tva^{delCCGCTG/delCCGCTG} may be obtained in the offsprings generated after the breeding of roosters and hens with both genotypes of tva^{s/delACCTCC} and/or tva^{s/delCCGCTG}, and the individuals are determined as chicken resistance to ALV-A and ALV-K.

Further, a PCR amplification system includes: 1 μL of a DNA template, 2.5 μL of a 10× buffer, 2 μL of dNTPs, 1 μL of each of upstream and downstream detection primers, 0.5 μL of KOD-FX, and supplementing to 25 μL with ddH₂O.

In some embodiments, a PCR amplification program includes: initial denaturation at 94° C. for 5 min; 30 cycles of denaturation at 94° C. for 30 sec, annealing at 58° C. for 30 sec, and extension at 72° C. for 30 sec; post extension at 72° C. for 5 min, and storage at 4° C.

The present disclosure further provides use of the molecular markers or the primers inbreeding of chicken resistant to ALV-A and ALV-K.

The present disclosure further provides a kit for detecting/screening chicken resistant to ALV-A and ALV-K, wherein the kit includes the primers.

The present disclosure further provides the use of the kit in breeding of the chicken resistant to ALV-A and ALV-K.

The technical scheme of the present disclosure has the following beneficial effects:

According to the present disclosure, it is found for the first time that the DNA sequence (with a GenBank accession number of AY531262.1) of a co-receptor gene tva of the ALV-A and ALV-K exists at base positions 318 to 323 or at base positions 602 to 607 (tva^{318-323delACCTCC} and tva^{602-607delCCGCTG}) in Chinese chicken breeds. Further researches have confirmed that natural mutation of tva gene can cause a host chicken to generate genetic resistance to ALV-A and ALV-K infection. Therefore, the mutation site can be used as a molecular marker for identifying the chickens genetic resistance to ALV-A and ALV-K.

In the present disclosure, a molecular diagnosis and genotyping method further established based on the molecular marker for genetic resistance to chicken ALV-A and ALV-K, tva^{318-323delACCTCC} or tva^{602-607delCCGCTG}. Furthermore, a method for identifying chicken resistant to ALV-A and ALV-K has been established, which is able to quickly and accurately determine whether a test sample is chicken resistant to or susceptible to ALV-A and ALV-K. The method can be applied to screen breeding materials of chicken breeds (lines) with genetic resistance to ALV-A and ALV-K in Chinese chicken breeds (including local chicken breeds and commercial chicken lines), thereby carrying out breeding of the chicken breeds (lines) with genetic resistance to ALV-A and ALV-K. The method has desirable application and promotion values.

### BRIEF DESCRIPTION OF THE DRAWINGS

FIG. 1 shows PCR amplification results of three fragments of tva gene; where M indicates DL2000 marker; 1 to 3 indicate PCR amplified products of primers 1, 2, and 3, respectively;

FIG. 2 is a sequencing map showing different genotype sequences of the tva^{318-323delACCTCC} site, including the wild type (SEQ ID NO:16), heterozygous mutant (SEQ ID NO:17) and the homozygous mutant (SEQ ID NO:18) sequences;

FIG. 3 is a sequencing map showing different genotype sequences of the tva^{602-607delCCGCTG} site, including the wild type (SEQ ID NO:13), heterozygous mutant (SEQ ID NO:14) and the homozygous mutant (SEQ ID NO:15) sequences;

FIG. 4A-FIG. 4C show a schematic diagram of construction of RCASBP(A)-EGFP and RCASBP(K)-EGFP expression plasmids and their rescue of fluorescent reporter virus; where FIG. 4A indicates a schematic diagram of the construction of RCASBP(A)-EGFP and RCASBP(K)-EGFP expression plasmids; FIG. 4B indicates enzyme digestion identification of the RCASBP(A)-EGFP and RCASBP(K)-EGFP plasmids; and FIG. 4C indicates the rescue of RCASBP(A)-EGFP and RCASBP(K)-EGFP virus;

FIG. 5 shows a process of RCASBP(A)-EGFP virus infecting CEF cells with different genotypes at the tva^{318-323delACCTCC} site;

FIG. 6 shows a process of RCASBP(K)-EGFP virus infecting CEF cells with different genotypes at the tva^{318-323delACCTCC} site;

FIG. 7 shows a situation of RCASBP(A)-EGFP virus infecting CEFs with different genotypes at the tva^{602-607delCCGCTG} mutation site; and

FIG. 8 shows a situation of RCASBP(K)-GFP virus infecting CEFs with different genotypes at the tva^{602-607delCCGCTG} mutation site.

### DETAILED DESCRIPTION OF THE EMBODIMENTS

The specific embodiments of the present disclosure are described below to facilitate those skilled in the art to understand the technical scheme of the present disclosure, but it should be clear that the present disclosure is not limited to the scope of the specific embodiments. Various obvious changes made by those of ordinary skill in the art within the spirit and scope of the present disclosure defined by the attached claims should fall within the protection scope of the present disclosure.

### Example 1 Screening of Tva^{318-323delACCTCC} Molecular Marker

1. Primers Design for PCR Amplification of Tva Receptor Gene

Referring to the DNA sequence of chicken tva gene in NCBI database (with a GenBank accession number of AY531262.1), 3 pairs of primers were designed to amplify the full-length sequence of tva gene (3607 bp) by PCR in three fragments (fragments 1, 2 and 3), primer sequences, positions, and sizes of PCR amplified fragments were shown in Table 1.

TABLE 1
PCR amplification information of full-length sequence of tva receptor gene

| | | | tva gene | Target | |
|---|---|---|---|---|---|
| | Primer | | sequence | fragment | |
| Fragment | name | Primer sequence (5′-3′) | position | size (bp) | |
| Fragment | P1-F | GTTCAGCAGATCCTCAT | 17-39 | 1308 | SEQ ID NO: 7 |
| 1 | | CTCCCG | | | |
| | P1-R | GGCCATTGTGCGATCTA | 1302-1324 | | SEQ ID NO: 8 |
| | | AGAGGG | | | |
| Fragment | P2-F | AGCCCTCTTAGATCGCA | 1300-1319 | 1253 | SEQ ID NO: 9 |
| 2 | | CAA | | | |
| | P2-R | GTGACACCGAGCACAA | 2533-2552 | | SEQ ID NO: 10 |
| | | AATG | | | |
| Fragment | P3-F | GTTGGAGCTGGATGAG | 2464-2483 | 1132 | SEQ ID NO: 11 |
| 3 | | CACT | | | |
| | P3-R | TGAGGGAATTCCTGTCA | 3576-3595 | | SEQ ID NO: 12 |
| | | CCT | | | |

2. PCR Amplification of Tva Receptor Gene

(1) Genomic DNAs of 6570 blood samples were extracted from different Chinese chicken breeds (including 28 local chicken breeds and 57 yellow feathered broiler lines), and the full-length sequence of tva gene was amplified by PCR with the 3 pairs of primers.

The PCR amplification system included: 1 μL of a DNA template, 2.5 μL of a 10× buffer, 2 μL of dNTPs, 1 μL of each of upstream and downstream primers, 0.5 μL of KOD-FX, and supplementing to 25 μL with ddH₂O.

The PCR amplification program included: initial denaturation at 94° C. for 3 min; 35 cycles of denaturation at 94° C. for 30 sec, annealing (fragment 1 at 62° C., fragments 2 and 3 at 60° C.) for 30 sec, and extension at 72° C. for 90 sec; then post extension at 72° C. for 10 min, and storage at 4° C.

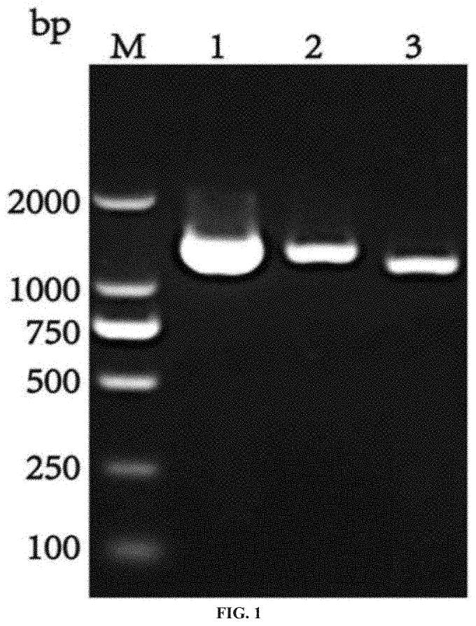

(2) PCR products were detected by 2% agarose gel electrophoresis, and the results were shown in FIG. 1; where M: DL2000 marker; 1 to 3: PCR amplified products of primers 1, 2, and 3. As shown in FIG. 1, target bands of the fragments 1, 2 and 3 of tva gene were amplified by PCR, with fragment sizes consistent with expected results.

(3) The PCR amplified products were sent to Sangon Biotech (Shanghai) Co., Ltd. for purification and sequencing, sequence comparison was conducted by DNAstar and Mutation Surveyor gene sequence analysis software, genetic variation of tva receptor gene in Chinese chicken breeds was analyzed, and candidate genetic resistance loci were screened for ALV-A and ALV-K.

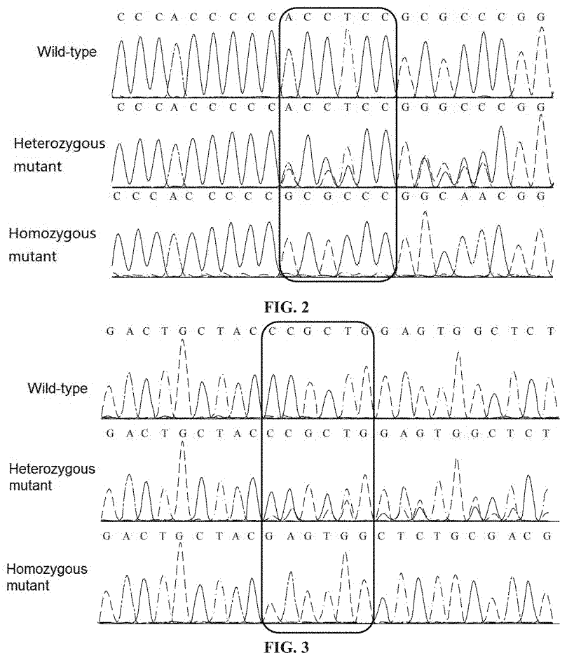

By analyzing the genetic variation of tva receptor gene from 28 local chicken breeds and 57 yellow feathered broiler lines (a total of 6570 blood samples), a natural mutation of ACCTCC base sequence deletion (tva^{318-323delACCTCC}) at positions 318-323 of tva receptor gene sequence of the Chinese chicken breeds were selected and found. The sequence sequencing map was shown in FIG. 2 (in FIG. 2, sequences from top to bottom were reference sequence (wild-type individual), sequence of heterozygous mutant individual, and sequence of homozygous mutant individual successively, in which the box showed the ACCTCC deletion mutation at positions 318 to 323 of tva gene sequence).

### Example 2 Screening of Tva^{602-607delCCGCTG} Molecular Marker

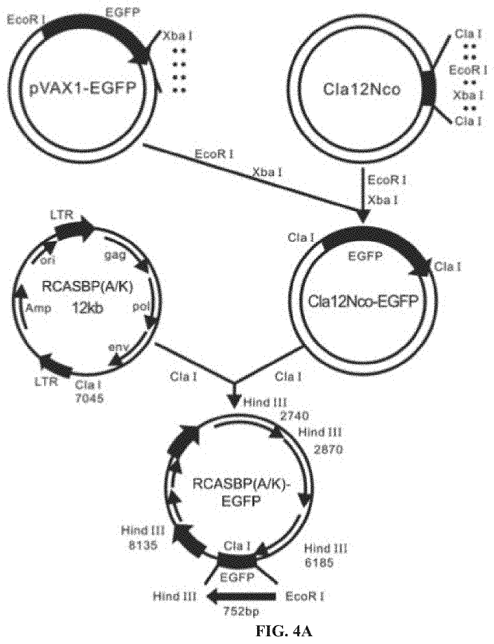

The screening process was the same as that of Example 1. The PCR amplified products were sent to Sangon Biotech (Shanghai) Co., Ltd. for purification and sequencing, sequence comparison was conducted by DNAstar and Mutation Surveyor gene sequence analysis software, genetic variation of tva receptor gene in Chinese chicken breeds was analyzed, and candidate genetic resistance loci were screened for ALV-A and ALV-K, as shown in FIG. 3. In FIG. 3, sequences from top to bottom were reference sequence (wild-type individual), sequence of heterozygous mutant individuals, and sequence of homozygous mutant individuals successively, in which the box showed the CCGCTG deletion mutation at positions 602 to 607 of tva gene sequence.

As shown in FIG. 3, by analyzing the genetic variation of tva receptor gene from 28 local chicken breeds and 57 yellow feathered broiler lines (a total of 6570 blood samples), natural mutation of CCGCTG base sequence deletion (tva^{602-607delCCGCTG}) at positions 602-607 of tva receptor gene sequence of the Chinese chicken breeds were selected and found.

### Example 3 Effects of Tva^{318-323delACCTCC} Mutation on Host Resistance

1. In Vitro Cell Experiments

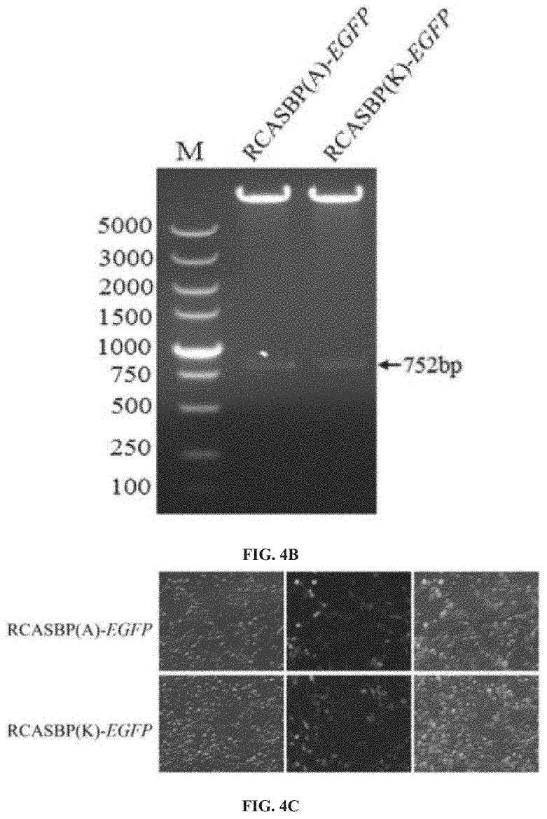

(1) RCASBP(A)-EGFP and RCASBP(K)-EGFP expression plasmids were constructed and transfected into DF-1 cells; 7 days after the transfection, RCASBP(A)-EGFP and RCASBP(K)-EGFP viruses (namely ALV-A and ALV-K reporter viruses carrying EGFPs) were rescued and collected from a supernatant of the DF-1 cells (FIG. 4A-FIG. 4C).

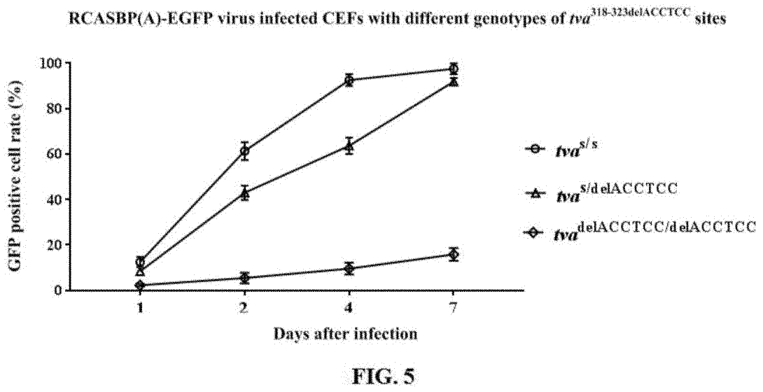

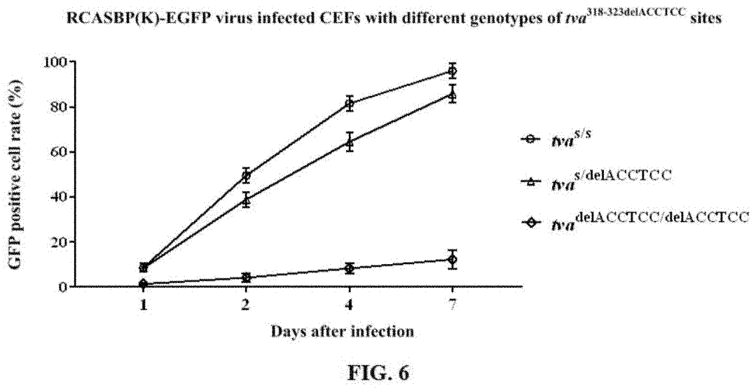

(2) The fluorescent reporter viruses RCASBP(A)-EGFP and RCASBP(K)-EGFP of ALV-A and ALV-K were used to infect chicken embryo fibroblasts (CEFs) at the tva^{318-323delACCTCC} mutation sites of the wild-type tva^{s/s}, the heterozygous mutant tva^{s/delACCTCC}, and the homozygous mutant tva^{delACCTCC/delACCTCC} separately (where the CEFs were prepared from 9-day-old chicken embryos hatched from the breeders tested in Example 1); 1 day, 2 days, 4 days, and 7 days after the infection, the CEFs with different genotypes of tva^{318-323delACCTCC} mutation sites infected by the RCASBP(A)-EGFP and RCASBP(K)-EGFP viruses were detected using flow cytometry; a GPF-positive cell rate (%) represented an infection rate of the virus, and results were shown in FIG. 5 and FIG. 6.

As shown in FIG. 5 and FIG. 6, the CEFs of wild-type tva^{s/s} and heterozygous mutant tva^{s/delACCTCC} were susceptible to the RCASBP(A)-EGFP and RCASBP(K)-EGFP viruses; while the CEFs of homozygous mutant tva^{delACCTCC/delACCTCC} were resistant to the RCASBP(A)-EGFP and RCASBP(K)-EGFP viruses. This indicated that the natural mutation of tva^{318-323delACCTCC} led to the host resistance to ALV-A, ALV-K infection.

2. In Vivo Experiments

(1) 1-day-old chicks with tva^{318-323delACCTCC} of the mutant wild type, heterozygous mutant, and homozygous mutant were randomly divided into groups, reared in isolators, and injected intraperitoneally with equal amounts of ALV-A (GD08 strain) and ALV-K (GDFX0601 strain) separately at 1-day-old and 5-day-old. One month after challenge, blood samples were collected from the chicks and a total RNA was extracted from each blood sample using a TRIZOL kit.

The upstream and downstream primers were designed for RT-PCR amplification of ALV-A-env:

env-F:
(SEQ ID NO: 3)
5′-GGATGAGGTGACTAAGAAAG-3′;
env-R:
(SEQ ID NO: 4)
5′-AGAGAAAGAGGGGTGTCTAAGGAGA-3′.

(2) The encoding sequence of env gene of ALV-A was amplified by RT-PCR, and the amplified fragment by RT-PCR had a length of 692 bp. RT-PCR amplification was conducted by the PrimeScript® One Step RT-PCR Kit Ver. 2, and the PCR reaction program included: reverse transcription at 50° C. for 30 min; at 94° C. for 30 sec, at 56° C. for 30 sec, and at 72° C. for 60 sec, conducting 35 cycles; and post extension at 72° C. for 10 min. PCR products were detected by 2% agarose gel electrophoresis; if a 692 bp target band was observed, the sample was infected with viremia (ALV-A positive); if there was no amplified target band, the sample was not infected with viremia (ALV-A negative), as shown in Table 4.

(3) The upstream and downstream primers were designed for RT-PCR amplification of ALV-K-env:

env-F:
(SEQ ID NO: 5)
5′- GCACCACCTTGGGAACTGACC-3′;
env-R:
(SEQ ID NO: 6)
5′-GGCGTGGATCGACAGCACAC-3′.

The encoding sequence of env gene of ALV-K was amplified by RT-PCR, and the amplified fragment by RT-PCR had a length of 633 bp. RT-PCR amplification was conducted by the PrimeScript® One Step RT-PCR Kit Ver. 2, and the PCR reaction program included: reverse transcription at 50° C. for 30 min; at 94° C. for 30 sec, at 60° C. for 30 sec, and at 72° C. for 60 sec, conducting 35 cycles; and post extension at 72° C. for 10 min. PCR products were detected by 2% agarose gel electrophoresis; if a 633 bp target band was observed, the sample was infected with viremia (ALV-K positive); if there was no amplified target band, the sample was not infected viremia (ALV-K negative), as shown in Table 5.

TABLE 2
Incidence of ALV-A infection in 1-day-old chicks with different
genotypes of tva^{318-323delACCTCC} mutation sites after being
challenged with wild ALV-A virus for 1 month

| | | Number of positive | Positive |
|---|---|---|---|
| tva gene | | samples/total number | infection |
| mutation site | Genotype | of samples | rate (%) |
| tva^{318-323delACCTCC} | Wild-type tva^{s/s} | 28/28 | 100 |
| | tva^{s/delACCTCC} | 25/25 | 100 |
| | tva^{delACCTCC/delACCTCC} | 0/18 | 0 |

TABLE 3
Incidence of ALV-K infection in 1-day-old chicks with different
genotypes of tva^{318-323delACCTCC} mutation sites after being
challenged with wild ALV-K virus for 1 month

| | | Number of positive | Positive |
|---|---|---|---|
| tva gene | | samples/total number | infection |
| mutation site | Genotype | of samples | rate (%) |
| tva^{318-323delACCTCC} | Wild-type tva^{s/s} | 28/28 | 100 |
| | tva^{s/delACCTCC} | 25/25 | 100 |
| | tva^{delACCTCC/delACCTCC} | 0/18 | 0 |

As shown in Table 2 and Table 3, the wild-type tva^{s/s} chicks for the tva^{318-323delACCTCC} mutation sites (28) each showed ALV-A and ALV-K positive after being challenged with ALV-A and ALV-K wild viruses; the heterozygous mutant tva^{s/delACCTCC} chicks (25) each showed ALV-A and ALV-K positive after being challenged with ALV-A and ALV-K wild viruses; however, the homozygous mutant tva^{delACCTCC/delACCTCC} chicks (18) each showed ALV-A and ALV-K negative after being challenged with ALV-A and ALV-K wild viruses. The experimental results showed that the natural mutation of tva^{318-323delACCTCC} led to the host resistance to ALV-A and ALV-K infection in vivo. The results of the ALV-A and ALV-K challenge tests were consistent with the results of the ALV-A and ALV-K in vitro infection tests. Meanwhile, it was confirmed that the natural mutation of tva^{318-323delACCTCC} was the molecular marker for genetic resistance to ALV-A and ALV-K in the host chicken.

### Example 4 Effects of Tva^{602-607delCCGCTG} Mutation on Host Resistance

1. In Vitro Cell Experiments

(1) RCASBP(A)-EGFP and RCASBP(K)-EGFP expression plasmids were constructed and transfected into DF-1 cells; 7 days after the transfection, RCASBP(A)-EGFP and RCASBP(K)-EGFP viruses (namely ALV-A and ALV-K reporter viruses carrying EGFPs) were rescued and collected from the supernatant of the DF-1 cells (FIG. 4A-FIG. 4C); after measuring the viral infectious unit (IU), the supernatant was aliquoted and stored at −80° C.

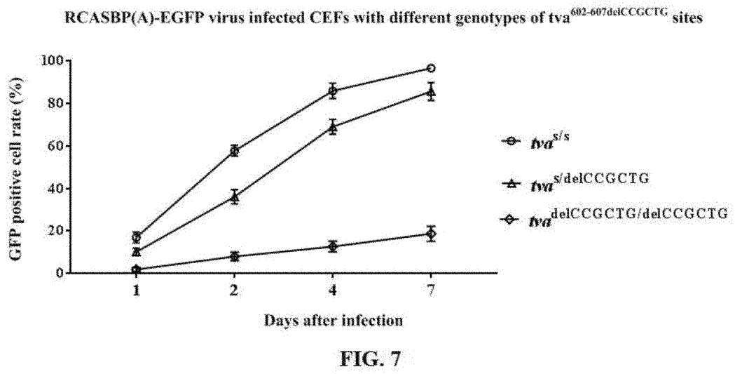

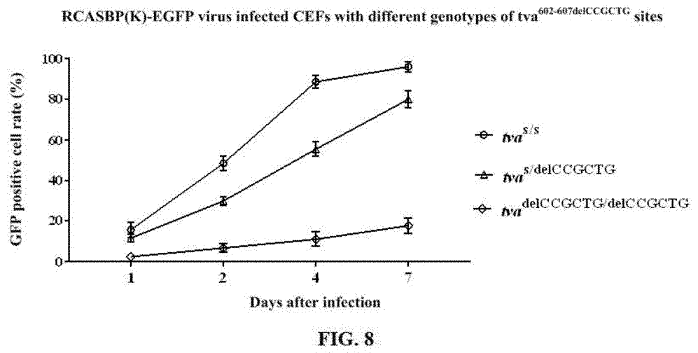

(2) The fluorescent reporter viruses RCASBP(A)-EGFP and RCASBP(K)-EGFP of ALV-A and ALV-K were used to infect CEFs at the tva^{602-607delCCGCTG} mutation sites of the wild-type tva^{s/s}, the heterozygous mutant tva^{s/delCCGCTG}, and the homozygous mutant tva^{delCCGCTG/delCCGCTG} separately (where the CEFs were prepared from 9-day-old chicken embryos hatched from the breeders tested in Example 1); 1 day, 2 days, 4 days, and 7 days after the infection, the CEFs with different genotypes of tva^{602-607delCCGCTG} mutation sites infected by the RCASBP(A)-EGFP and RCASBP(K)-EGFP viruses were detected using flow cytometry; the GPF-positive cell rate (%) represented the infection rate of the virus, and results were shown in FIG. 7 and FIG. 8.

As shown in FIG. 7 and FIG. 8, the CEFs of wild-type tva^{s/s} and heterozygous mutant tva^{s/delCCGCTG} were susceptible to the RCASBP(A)-EGFP and RCASBP(K)-EGFP viruses; while the CEFs of homozygous mutant tva^{delCCGCTG/delCCGCTG} were resistant to the RCASBP(A)-EGFP and RCASBP(K)-EGFP viruses. This indicated that the natural mutation of tva^{602-607delCCGCTG} led to the host resistance to ALV-A, ALV-K infection.

2. In Vivo Experiments

(1) 1-day-old chicks with tva^{602-607delCCGCTG} of the mutant wild type, heterozygous mutant, and homozygous mutant were randomly divided into groups, reared in isolators, and injected intraperitoneally with equal amounts of ALV-A (GD08 strain) and ALV-K (GDFX0601 strain) separately at 1-day-old and 5-day-old. One month after challenge, blood samples were collected from the chicks and the total RNA was extracted from each blood sample using the TRIZOL kit.

The upstream and downstream primers were designed for RT-PCR amplification of ALV-A-env:

env-F:
(SEQ ID NO: 3)
5′-GGATGAGGTGACTAAGAAAG-3′;
env-R:
(SEQ ID NO: 4)
5′-AGAGAAAGAGGGGTGTCTAAGGAGA-3′.

(2) The encoding sequence of env gene of ALV-A was amplified by RT-PCR, and the amplified fragment by RT-PCR had a length of 692 bp. RT-PCR amplification was conducted by the PrimeScript® One Step RT-PCR Kit Ver. 2, and the PCR reaction program included: reverse transcription at 50° C. for 30 min; at 94° C. for 30 sec, at 56° C. for 30 sec, and at 72° C. for 60 sec, conducting 35 cycles; and post extension at 72° C. for 10 min. PCR products were detected by 2% agarose gel electrophoresis; if a 692 bp target band was observed, the sample was infected with viremia (ALV-A positive); if there was no amplified target band, the sample was not infected with viremia (ALV-A negative), as shown in Table 2.

(3) The upstream and downstream primers were designed for RT-PCR amplification of ALV-K-env:

env-F:
(SEQ ID NO: 5)
5′- GCACCACCTTGGGAACTGACC-3′;
env-R:
(SEQ ID NO: 6)
5′-GGCGTGGATCGACAGCACAC-3′.

The encoding sequence of env gene of ALV-K was amplified by RT-PCR, and the amplified fragment by RT-PCR had a length of 633 bp. RT-PCR amplification was conducted by the PrimeScript® One Step RT-PCR Kit Ver. 2, and the PCR reaction program included: reverse transcription at 50° C. for 30 min; at 94° C. for 30 sec, at 60° C. for 30 sec, and at 72° C. for 60 sec, conducting 35 cycles; and post extension at 72° C. for 10 min. PCR products were detected by 2% agarose gel electrophoresis; if a 633 bp target band was observed, the sample was infected with viremia (ALV-K positive); if there was no amplified target band, the sample was not infected with viremia (ALV-K negative), as shown in Table 3.

TABLE 4
Incidence of ALV-A infection in 1-day-old chicks with different
genotypes of tva^{602-607delCCGCTG} mutation sites after being
challenged with wild ALV-A virus for 1 month

| | | Number of positive | Positive |
|---|---|---|---|
| tva gene | | samples/total number | infection |
| mutation site | Genotype | of samples | rate (%) |
| tva^{602-607delCCGCTG} | Wild-type tva^{s/s} | 32/32 | 100 |
| | tva^{s/delCCGCTG} | 23/23 | 100 |
| | tva^{delCCGCTG/deICCGCTG} | 0/27 | 0 |

TABLE 5
Incidence of ALV-K infection in 1-day-old chicks with different
genotypes of tva^{602-607delCCGCTG} mutation sites after being
challenged with wild ALV-K virus for 1 month

| | | Number of positive | Positive |
|---|---|---|---|
| tva gene | | samples/total number | infection |
| mutation site | Genotype | of samples | rate (%) |
| tva^{602-607delCCGCTG} | Wild-type tva^{s/s} | 32/32 | 100 |
| | tva^{s/delCCGCTG} | 23/23 | 100 |
| | tva^{delCCGCTG/delCCGCTG} | 0/27 | 0 |

As shown in Table 4 and Table 5, at the tva^{602-607delCCGCTG} mutation sites, the wild-type tva^{s/s} chicks (32) each showed ALV-A and ALV-K positive after being challenged with ALV-A and ALV-K wild viruses; the heterozygous mutant tva^{s/delCCGCTG} chicks (23) each showed ALV-A and ALV-K positive after being challenged with ALV-A and ALV-K wild viruses; however, the homozygous mutant tva^{delCCGCTG/delCCGCTG} chicks (27) each showed ALV-A and ALV-K negative after being challenged with ALV-A and ALV-K wild viruses. The experimental results showed that the natural mutation of tva^{602-607delCCGCTG} led to the host resistance to ALV-A and ALV-K infection in vivo. The results of the ALV-A and ALV-K challenge tests were consistent with the results of the ALV-A and ALV-K in vitro infection tests. Meanwhile, it was confirmed that the natural mutation of tva^{602-607delCCGCTG} was a molecular marker for genetic resistance to ALV-A and ALV-K in the host chicken.

### Example 5 Screening of Chickens with Genetic Resistance to ALV-A and ALV-K

1. Referring to a DNA sequence of tva gene (with a GenBank accession number of AY531262.1), PCR primers were designed (including a forward primer F: 5′-CGGCCCGCTTTATAGGCGTTG-3′ (SEQ ID NO: 1); a reverse primer R: 5′-CCCACTCGTCCCGTCCATCG-3′ (SEQ ID NO: 2)), and a tva receptor gene region containing tva^{602-607delCCGCTG} or tva^{318-323delACCTCC} mutation sites was amplified.

2. The genomic DNAs were extracted from 1782 samples to be tested of 15 local chicken breeds and 15 yellow feathered broiler lines.

3. PCR detection

The PCR amplification system included: 1 μL of a DNA template, 2.5 μL of a 10× buffer, 2 μL of dNTPs, 1 μL of each of upstream and downstream detection primers (with a nucleotide sequence set forth in SEQ ID NO: 1), 0.5 μL of KOD-FX, and supplementing to 25 μL with ddH₂O.

The PCR amplification program included: initial denaturation at 94° C. for 5 min; denaturation at 94° C. for 30 sec, annealing at 58° C. for 30 sec, and extension at 72° C. for 30 sec, conducting 35 cycles; then post extension at 72° C. for 5 min, and storage at 4° C.

4. After detection by 2% agarose gel electrophoresis, the PCR amplified products were sent to Sangon Biotech (Shanghai) Co., Ltd. for purification and sequencing to determine the genotype and whether the sample to be tested was a resistant chicken. The determination criteria were shown in Table 6 and Table 7.

TABLE 6
Identification criteria for chicken with
genetic resistance to ALV-A and ALV-K

| tva gene | | Susceptibility to ALV-A |
|---|---|---|
| mutation site | Genotype | and ALV-K infection |
| tva^{602-607delCCGCTG} | Wild-type tva^{s/s} | Susceptible |
| | tva^{s/delCCGCTG} | Susceptible |
| | tva^{delCCGCTG/delCCGCTG} | Resistant |

TABLE 7
Identification criteria for chicken with
genetic resistance to ALV-A and ALV-K

| tva gene | | Susceptibility to ALV-A |
|---|---|---|
| mutation site | Genotype | and ALV-K infection |
| tva^{318-323delACCTCC} | Wild-type tva^{s/s} | Susceptible |
| | tva^{s/delACCTCC} | Susceptible |
| | tva^{delACCTCC/delACCTCC} | Resistant |

If the genotype of the tva^{602-607delCCGCTG} or tva^{318-323delACCTCC} resistance site was wild-type tva^{s/s}, the sample to be tested was not resistant to ALV-A and ALV-K infection (susceptible), such that the sample to be tested was determined to be the chicken susceptible to ALV-A and ALV-K;

if the genotype of the tva^{602-607delCCGCTG} resistance site was tva^{s/delCCGCTG} or the genotype of the tva^{318-323delACCTCC} resistance site was tva^{s/delACCTCC}, the sample to be tested was susceptible to ALV-A and ALV-K infection, but the sample to be tested carried the recessive gene for the genetic resistance of ALV-A and ALV-K; and

if the genotype of the tva^{602-607delCCGCTG} resistance site was tva^{delCCGCTG/delCCGCTG} or the genotype of the tva^{318-323delACCTCC} resistance site was tva^{delACCTCC/delACCTCC}, the sample to be tested was resistant to ALV-A and ALV-K infection, such that the sample to be tested was determined to be the chicken resistant to ALV-A and ALV-K.

5. Test results

In Chinese chicken breeds, the genotyping results of tva^{318-323delACCTCC} resistance site were shown in Table 8, and the genotyping results of tva^{602-607delCCGCTG} resistance site were shown in Table 9.

As shown in Table 8, Broiler chicken, Lingshan native chicken, Xuefeng silky chicken, and Xiushui yellow chicken and other local chicken breeds, as well as yellow feathered broiler line 1, yellow feathered broiler line 4, yellow feathered broiler line 10, and yellow feathered broiler line 12 had the resistance genotype tva^{delACCTCC/delACCTCC} for the tva^{318-323delACCTCC} resistance site at frequencies of 0.10, 0.33, 0.15, 0.12, 0.20, 0.25, 0.25, and 0.18, respectively. This indicated that these Chinese local chicken breeds and self-bred yellow feathered broiler lines had desirable potential for genetic improvement against ALV-A and ALV-K. Breeding materials for cultivating breeds resistant to ALV-A and ALV-K infection can be screened from these chicken breeds, and used in the breeding of chicken breeds (lines) with genetic resistance to ALV-A and ALV-K, to control the subgroups A and K-caused AL.

As shown in Table 9, Huaixiang chicken, Hetian chicken, and Chongren ma chicken and other local chicken breeds, as well as yellow feathered broiler line 2, yellow feathered broiler line 5, yellow feathered broiler line 11, and yellow feathered broiler line 14 had the resistance genotype tva^{delCCGCTG/delCCGCTG} for the tva^{602-607delCCGCTG} resistance site at frequencies of 0.07, 0.11, 0.17, 0.27, 0.10, 0.20, and 0.13, respectively. This indicated that these Chinese local chicken breeds and self-bred yellow feathered broiler lines had desirable potential for genetic improvement against ALV-A and ALV-K. Breeding materials for cultivating breeds resistant to ALV-A and ALV-K infection can be screened from these chicken breeds, and used in the breeding of chicken breeds (lines) with genetic resistance of ALV-A and ALV-K, to control the ALV subgroups A and K.

TABLE 8
Genotype frequency distribution of tva^{318-323delACCTCC} mutation sites in Chinese chicken breeds

| | Sample numbers | tva^{318-323delACCTCC} | | |
|---|---|---|---|---|
| Breeds (lines) | (chicken) | tva^{s/s} | tva^{s/delACCTCC} | tva^{delACCTCC/delACCTCC} |
| Huaixiang chicken | 60 | 0.92 | 0.08 | 0 |
| Zhongshan Shalan chicken | 60 | 1 | 0 | 0 |
| Beijing Fatty chicken | 50 | 1 | 0 | 0 |
| Broiler chicken | 60 | 0.77 | 0.13 | 0.10 |
| Longsheng Fengji chicken | 58 | 1 | 0 | 0 |
| Lingshan native chicken | 36 | 0.56 | 0.11 | 0.33 |
| Taihe silky chicken | 50 | 1 | 0 | 0 |
| Hetian chicken | 72 | 1 | 0 | 0 |
| Xuefeng silky chicken | 60 | 0.62 | 0.23 | 0.15 |
| Wenchang chicken | 60 | 1 | 0 | 0 |
| Baier huang chicken | 60 | 1 | 0 | 0 |
| Chongren ma chicken | 60 | 1 | 0 | 0 |
| Anyiwa gray chicken | 60 | 1 | 0 | 0 |
| Xiushui yellow chicken | 50 | 0.72 | 0.16 | 0.12 |
| Xiayan chicken | 50 | 1 | 0 | 0 |
| Yellow feathered broiler line 1 | 60 | 0.55 | 0.25 | 0.20 |
| Yellow feathered broiler line 2 | 72 | 1 | 0 | 0 |
| Yellow feathered broiler line 3 | 72 | 1 | 0 | 0 |
| Yellow feathered broiler line 4 | 60 | 0.45 | 0.30 | 0.25 |
| Yellow feathered broiler line 5 | 60 | 1 | 0 | 0 |
| Yellow feathered broiler line 6 | 60 | 0.85 | 0.15 | 0 |
| Yellow feathered broiler line 7 | 60 | 1 | 0 | 0 |
| Yellow feathered broiler line 8 | 60 | 1 | 0 | 0 |
| Yellow feathered broiler line 9 | 60 | 1 | 0 | 0 |
| Yellow feathered broiler line 10 | 60 | 0.53 | 0.22 | 0.25 |
| Yellow feathered broiler line 11 | 60 | 1 | 0 | 0 |
| Yellow feathered broiler line 12 | 60 | 0.54 | 0.28 | 0.18 |
| Yellow feathered broiler line 13 | 60 | 1 | 0 | 0 |
| Yellow feathered broiler line 14 | 60 | 1 | 0 | 0 |
| Yellow feathered broiler line 15 | 60 | 1 | 0 | 0 |

TABLE 9
Genotype frequency distribution of tva602-607delCCGCTG
mutation sites in Chinese chicken breeds

| | Sample numbers | tva^{602-607delCCGCTG} | | |
|---|---|---|---|---|
| Breeds (lines) | (chicken) | tva^{s/s} | tva^{s/delCCGCTG} | tva^{delCCGCTG/delCCGCTG} |
| Huaixiang chicken | 60 | 0.77 | 0.16 | 0.07 |
| Zhongshan Shalan chicken | 60 | 1 | 0 | 0 |
| Beijing Fatty chicken | 50 | 1 | 0 | 0 |
| Broiler chicken | 60 | 1 | 0 | 0 |
| Longsheng Fengji chicken | 58 | 1 | 0 | 0 |
| Lingshan native chicken | 36 | 1 | 0 | 0 |
| Taihe silky chicken | 50 | 1 | 0 | 0 |
| Hetian chicken | 72 | 0.56 | 0.33 | 0.11 |
| Xuefeng silky chicken | 60 | 1 | 0 | 0 |
| Wenchang chicken | 60 | 1 | 0 | 0 |
| Baier huang chicken | 60 | 1 | 0 | 0 |
| Chongren ma chicken | 60 | 0.60 | 0.23 | 0.17 |
| Anyiwa gray chicken | 60 | 1 | 0 | 0 |
| Xiushui yellow chicken | 50 | 1 | 0 | 0 |
| Xiayan chicken | 50 | 1 | 0 | 0 |
| Yellow feathered broiler line 1 | 60 | 1 | 0 | 0 |
| Yellow feathered broiler line 2 | 72 | 0.43 | 0.30 | 0.27 |
| Yellow feathered broiler line 3 | 72 | 1 | 0 | 0 |
| Yellow feathered broiler line 4 | 60 | 1 | 0 | 0 |
| Yellow feathered broiler line 5 | 60 | 0.60 | 0.30 | 0.10 |
| Yellow feathered broiler line 6 | 60 | 0.83 | 0.17 | 0 |
| Yellow feathered broiler line 7 | 60 | 1 | 0 | 0 |
| Yellow feathered broiler line 8 | 60 | 1 | 0 | 0 |
| Yellow feathered broiler line 9 | 60 | 1 | 0 | 0 |
| Yellow feathered broiler line 10 | 60 | 1 | 0 | 0 |
| Yellow feathered broiler line 11 | 60 | 0.63 | 0.17 | 0.20 |
| Yellow feathered broiler line 12 | 60 | 1 | 0 | 0 |
| Yellow feathered broiler line 13 | 60 | 1 | 0 | 0 |
| Yellow feathered broiler line 14 | 60 | 0.4 | 0.47 | 0.13 |
| Yellow feathered broiler line 15 | 60 | 0.93 | 0.07 | 0 |

1. A molecular marker for genetic resistance of chicken to infection by subgroups A and K avian leukosis virus (ALV-A and ALV-K), wherein the molecular marker is tva gene with base deletion between 318-323 and/or between 602-607;
wherein, bases ACCTCC at positions 318-323 and bases CCGCTG at positions 602-607 are deleted; and
a GenBank accession number of tva gene is AY531262.1.

2. The molecular marker for genetic resistance of chicken to infection by ALV-A and ALV-K according to claim 1, wherein the molecular marker is a DNA sequence of tva gene with base deletion of ACCTCC at positions 318-323.

3. The molecular marker for genetic resistance of chicken to infection by ALV-A and ALV-K according to claim 1, wherein the molecular marker is a DNA sequence of tva gene with base deletion of CCGCTG at positions 602-607.

4-10. (canceled)
